(19) United States
(12) Patent Application Publication
(10) Pub. No.: US 2024/0042226 A1
(43) Pub. Date: Feb. 8, 2024

(54) TRANSCRANIAL MAGNETIC STIMULATOR

(71) Applicant: Teijin Pharma Limited, Tokyo (JP)

(72) Inventors: Hitoshi NAKAMURA, Tokyo (JP); Kenji NANDOH, Matsudo-shi, Chiba (JP)

(73) Assignee: Teijin Pharma Limited, Tokyo (JP)

(21) Appl. No.: 18/266,956

(22) PCT Filed: Dec. 9, 2021
(86) PCT No.: PCT/JP2021/045288

§ 371 Date: Jun. 13, 2023

(30) Foreign Application Priority Data
Dec. 16, 2020 (JP) 2020-208513

(51) Int. Cl. A61N 2/00; A61N 2/02

(52) U.S. Cl. CPC A61N 2/006; A61N 2/02

(57) ABSTRACT

Provided is a transcranial magnetic stimulator capable of providing a magnetic stimulation with a required intensity inside a brain even when a current value and a voltage value applied to magnetic stimulation coils in a plurality of resonant circuits are reduced. The transcranial magnetic stimulator includes a plurality of resonant circuits for applying respective pulse currents to a plurality of magnetic stimulation coils to generate variable magnetic fields, and a power source that supplies an electric power to the plurality of resonant circuits. The plurality of resonant circuits are connected in parallel to the power source, and therefore, the plurality of magnetic stimulation coils are also connected in parallel to the power source. The plurality of magnetic stimulation coils are formed in approximately a same shape, and adjacently disposed such that directions of magnetic fluxes generated by the applied pulse currents are matched.

### TECHNICAL FIELD

The present invention relates to a transcranial magnetic stimulator used for performing transcranial magnetic stimulation.

### BACKGROUND ART

Transcranial magnetic stimulation (TMS) is a method that causes a current inside a brain by electromagnetic induction, thereby stimulating neurons (see Patent Documents 1 to 5 below). With this method, a variable magnetic field is generated by applying an alternate current or a predetermined current waveform to a stimulation coil put on a head surface, an eddy current is induced by the variable magnetic field, and then, neurons can be stimulated by the eddy current. The transcranial magnetic stimulation is used for therapies of diseases, such as depression, Alzheimer's dementia, schizophrenia, neuropathic pain, and Parkinson's disease, and additionally, used for various clinical examinations and brain function studies. With the transcranial magnetic stimulation, a non-invasive magnetic stimulation can be provided to neurons inside a brain without performing a craniotomy.

Now, in a magnetic stimulator used for the conventional transcranial magnetic stimulation, an LC resonant circuit including a capacitor and a stimulation coil is used, and magnetic stimulation can be provided by generating a variable magnetic field with supply of an electric charge accumulated in the capacitor from a high-voltage power source to the stimulation coil at a required timing by turning on/off a switch disposed in the resonant circuit. Accordingly, a frequency of the current (pulse current) applied to the stimulation coil is a resonant frequency of the LC resonant circuit.

Here, in the conventional device, it is necessary to flow a pulse current of several kA in the stimulation coil for providing the magnetic stimulation with a required intensity, and the pulse voltage in the case becomes on the order of kV. Therefore, in the conventional device, a thyristor capable of dealing with high current and high voltage is used as a switching element (Patent Document 1 below). However, since the thyristor is expensive, the conventional device has a problem of the increase in manufacturing cost of the entire device.

Therefore, Patent Document 2 below proposes a technique in which an inductor with high inductance is used to reduce a current, and a relatively low-price Insulated Gate Bipolar Transistor (IGBT) is used instead of the thyristor. However, in this technique, since a voltage applied to a stimulation coil increases, and it is necessary to set a withstand voltage of the switching element to be high, there is a problem again that the cost of the switching element increases.

Patent Document 3 below proposes a technique in which a plurality of resonant circuits with stimulation coils are connected in parallel to a power source, and magnetic fields from a plurality of directions of the respective stimulation coils are combined at a single point in a deep region inside a brain, thereby allowing stimulation in the deep region inside the brain. However, in this technique, it is necessary to apply a high voltage and a high current to each of the resonant circuits. Additionally, in this technique, to achieve the stimulation to the deep region inside the brain, the stimulation coils corresponding to the respective resonant circuits are disposed at different positions and faced in various directions. Then, depending on the stimulation position assigned in the brain, any of the plurality of stimulation coils possibly needs to be disposed apart from the stimulation position. In this case, the magnetic field of the coil attenuates due to the distance, and a desired magnetic field fails to be irradiated on an irradiation position. A problem arises in that the further high voltage and high current are required to avoid this.

While Patent Document 4 below discloses a technique in which stimulation coils are connected in parallel to a power source and a charging capacitor, also in this technique, a high voltage and a high current are applied to a switching element.

Therefore, this technique does not contribute to solving the above-described problem.
Patent Document 1: JP2016-67789A
Patent Document 2: JP2010-528784A
Patent Document 3: JP2010-536496A
Patent Document 4: U.S. Pat. No. 7,367,936
Patent Document 5: WO2017/175685

### DISCLOSURE OF THE INVENTION

### Problems to be Solved by the Invention

The present invention has been made based on the above-described circumstances. It is a main object of the present invention to provide a transcranial magnetic stimulator capable of providing a magnetic stimulation with a required intensity inside a brain even when a current value and a voltage value applied to magnetic stimulation coils in a plurality of resonant circuits are reduced. Another object of the present invention is to reduce a cost of an element, for example, a switching element, used for the resonant circuit by reducing the current value and the voltage value in the resonant circuit.

### Solutions to the Problems

The means for solving the above-described problems can be described as the following items.

(Item 1)

A transcranial magnetic stimulator includes a plurality of resonant circuits and a power source. The plurality of resonant circuits includes a plurality of magnetic stimulation coils for stimulating a living body by applying variable magnetic fields to an inside of the living body. The plurality of resonant circuits applies respective pulse currents to the plurality of magnetic stimulation coils to generate the variable magnetic fields. The power source supplies an electric power to the plurality of resonant circuits. The plurality of resonant circuits are connected in parallel to the power source, and therefore, the plurality of magnetic stimulation coils are also connected in parallel to the power source. The plurality of magnetic stimulation coils are formed in approximately a same shape, and adjacently disposed such that directions of magnetic fluxes generated by the pulse currents are matched.

(Item 2)

The transcranial magnetic stimulator according to Item 1, in which each of the plurality of resonant circuits includes a switching element that controls an application timing of the pulse current to the magnetic stimulation coil.

(Item 3)

The transcranial magnetic stimulator according to Item 1 or 2, in which the plurality of magnetic stimulation coils are disposed to be stacked such that axial centers of the plurality of magnetic stimulation coils are approximately matched.

(Item 4)

The transcranial magnetic stimulator according to Item 3, in which one of the magnetic stimulation coils is an upper coil, and another is a lower coil, and the upper coil and the lower coil are disposed to be stacked such that a bottom surface of the upper coil is overlapped with an upper surface of the lower coil in a cross-sectional view of at least a part of the upper coil and the lower coil.

(Item 5)

The transcranial magnetic stimulator according to Item 1 or 2, in which respective wound wires of the plurality of magnetic stimulation coils are mutually twisted, and form a multicore wire.

(Item 6)

The transcranial magnetic stimulator according to any one of Items 1 to 5, in which the resonant circuit includes a resonant capacitor that accumulates an electric charge supplied from the power source, a second switching element is disposed between the resonant capacitor and the power source, and the second switching element blocks a connection between the resonant capacitor and the power source during discharge of the resonant capacitor to suppress a leakage current to the power source.

(Item 7)

The transcranial magnetic stimulator according to any one of Items 1 to 5, in which a resonant impedance circuit is interposed between the resonant capacitor and the power source, and the resonant impedance circuit acts as a resistance component higher than a resistance component in non-resonance by resonating at a resonant frequency of the resonant circuit to suppress a leakage current to the power source.

(Item 8)

The transcranial magnetic stimulator according to any one of Items 1 to 7, in which any or all of the plurality of resonant circuits include a synchronization adjustment circuit for synchronizing the resonant frequency of each of the resonant circuits.

(Item 9)

The transcranial magnetic stimulator according to any one of Items 1 to 8, includes a phase adjustment circuit for matching phases of respective resonant currents generated in the plurality of resonant circuits.

(Item 10)

The transcranial magnetic stimulator according to Item 9, in which the phase adjustment circuit is configured to match the phases of the resonant currents by performing an adjustment so as to match generation timings of the respective resonant currents generated in the plurality of resonant circuits.

(Item 11)

The transcranial magnetic stimulator according to Item 9, in which the phase adjustment circuit is configured to match the phases of the resonant currents by performing an adjustment so as to match maximum points of change rates of the respective resonant currents generated in the plurality of resonant circuits.

(Item 12)

A transcranial magnetic stimulator includes a plurality of resonant circuits and a power source. The plurality of resonant circuits includes a plurality of magnetic stimulation coils for stimulating a living body by applying variable magnetic fields to an inside of the living body. The plurality of resonant circuits applies respective pulse currents to the plurality of magnetic stimulation coils to generate the variable magnetic fields. The power source supplies an electric power to the plurality of resonant circuits. The plurality of resonant circuits are connected in parallel to the power source, and therefore, the plurality of magnetic stimulation coils are also connected in parallel to the power source. The transcranial magnetic stimulator further includes a phase adjustment circuit for matching phases of respective resonant currents generated in the plurality of resonant circuits.

(Item 13)

The transcranial magnetic stimulator according to Item 12, in which the phase adjustment circuit is configured to match the phases of the resonant currents by performing an adjustment so as to match maximum points of change rates of the respective resonant currents generated in the plurality of resonant circuits.

### Effects of the Invention

According to the present invention, even when a current value and a voltage value applied to magnetic stimulation coils in a plurality of resonant circuits are reduced, by overlapping magnetic fluxes of the respective magnetic stimulation coils, a magnetic stimulation with a required intensity can be provided inside a brain. Accordingly, the current value and the voltage value of the resonant circuit can be reduced to be low, and consequently, the withstand voltage of the element, for example, a switching element used for the resonant circuit can be reduced. This also allows the reduction of the device cost.

### BRIEF DESCRIPTION OF THE DRAWINGS

FIG. 1 is a block diagram illustrating a schematic configuration of a transcranial magnetic stimulator according to a first embodiment of the present invention.

FIG. 2 is a circuit diagram for describing a circuit configuration in the transcranial magnetic stimulator of FIG. 1.

FIG. 3 is a schematic perspective view for describing an exemplary configuration of magnetic stimulation coils used for the circuit of FIG. 2.

FIG. 4 is an explanatory view for describing a stacked state of the magnetic stimulation coils disposed in an up-down direction, and a drawing corresponding to an end surface taken along the line A-A of FIG. 3.

FIG. 5(a) illustrates a time waveform of a pulse voltage applied to the magnetic stimulation coil, and FIG. 5(b) illustrates a time waveform of a pulse current flowing in the magnetic stimulation coil.

FIG. 6(a) is an explanatory view for describing an exemplary pattern of a magnetic stimulation to a living body, and FIG. 6(b) is an enlarged view of a part of FIG. 6(a).

FIG. 7 is an explanatory view for describing a configuration of magnetic stimulation coils used for a transcranial magnetic stimulator according to a second embodiment of the present invention, and an end view of a part corresponding to FIG. 4.

FIG. 8 is an explanatory view for describing a configuration of magnetic stimulation coils used for a transcranial magnetic stimulator according to a third embodiment of the present invention, and an end view of a part corresponding to FIG. 4.

FIG. 9 is an explanatory view illustrating an exemplary configuration of a multicore wire.

FIG. 10 is a circuit diagram in a transcranial magnetic stimulator according to a fourth embodiment of the present invention.

FIG. 11 is a drawing illustrating time waveforms of a voltage of a resonant capacitor and a current flowing in a magnetic stimulation coil.

FIG. 12 is a circuit diagram in a transcranial magnetic stimulator according to a fifth embodiment of the present invention.

FIG. 13 is a circuit diagram in a transcranial magnetic stimulator according to a sixth embodiment of the present invention.

FIG. 14 is a circuit diagram in a transcranial magnetic stimulator according to a seventh embodiment of the present invention.

FIG. 15 is a circuit diagram illustrating an example of a phase adjustment circuit used in the circuit of FIG. 14.

FIG. 16 is a circuit diagram illustrating another example of the phase adjustment circuit used in the circuit of FIG. 14.

FIG. 17 is a circuit diagram illustrating an example of a phase adjustment circuit used in a transcranial magnetic stimulator according to a seventh embodiment of the present invention.

FIG. 18 is a circuit diagram in a transcranial magnetic stimulator according to a ninth embodiment of the present invention.

FIG. 19 is a circuit diagram illustrating an example of a phase adjustment circuit used in the circuit of FIG. 18.

FIG. 20 is a circuit diagram illustrating an example of a phase adjustment circuit used in a transcranial magnetic stimulator according to a tenth embodiment of the present invention.

FIG. 21(a) illustrates an example of a current waveform applied to a magnetic stimulation coil in the circuit of FIG. 20, and FIG. 21(b) illustrates an exemplary waveform of an electric field generated by the current of FIG. 21(a).

### DESCRIPTION OF PREFERRED EMBODIMENTS

The following describes a transcranial magnetic stimulator (hereinafter simply referred to as a “stimulator” or a “device” in some cases) according to a first embodiment of the present invention with reference to the accompanying drawings. The device of the embodiment applies a variable magnetic field to an inside of a living body, thereby stimulating the living body, especially inside a brain.

### Configuration of the Embodiment

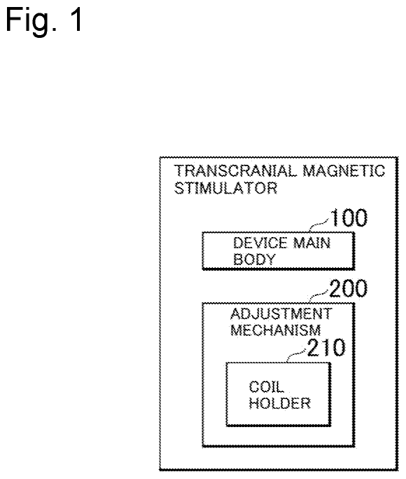

First, an example of a schematic configuration of the device according to the embodiment will be described with reference to FIG. 1. This device includes a device main body 100 and an adjustment mechanism 200. The device main body 100 supports the adjustment mechanism 200, and includes main equipment, such as a power source 3 described later. The adjustment mechanism 200 is configured to adjust a position of a coil holder 210 that holds magnetic stimulation coils 11, 12 described later, thereby providing a magnetic stimulation at a predetermined position of a head of a subject (not illustrated) seated on an appropriate chair (not illustrated). Since the device can have the overall configuration similar to a conventional one, the description in more detail will be omitted.

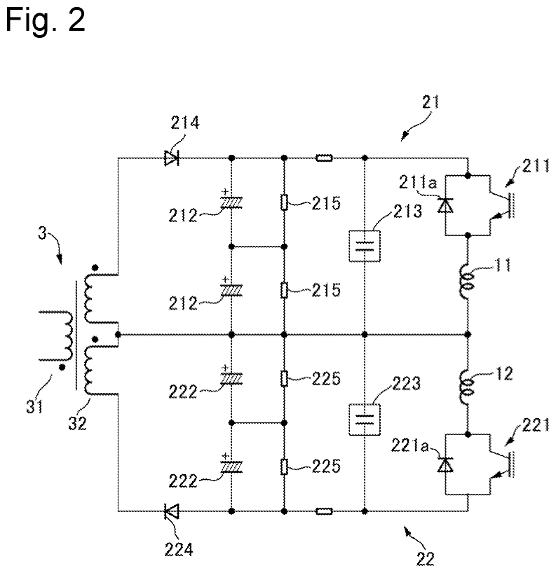

Next, with reference to FIG. 2, an exemplary configuration of a circuit for driving the magnetic stimulation coil 11, 12 in the device of this embodiment will be described. That is, the device includes a plurality of resonant circuits 21, 22 and a power source 3 as a basic configuration. The plurality of resonant circuits 21, 22 apply respective pulse currents to the plurality of magnetic stimulation coils 11, 12 for stimulating the living body by applying variable magnetic fields to an inside of a living body (specifically, inside brain) of a subject, thereby generating a variable magnetic field. The power source 3 supplies an electric power to the plurality of resonant circuits 21, 22.

(Resonant Circuit)

The plurality of resonant circuits 21, 22 are connected in parallel to the power source 3, and accordingly, the plurality of magnetic stimulation coils 11, 12 are also connected in parallel to the power source 3.

The plurality of resonant circuits 21, 22 include a plurality of switching elements 211 and 221 that control application timings of the pulse currents to the magnetic stimulation coils 11, 12, charging capacitors 212, 222 that accumulate electric charges supplied from the power source 3, resonant capacitors 213, 223 interposed in parallel to the magnetic stimulation coils 11, 12, rectifier diodes 214, 224, and resistors 215, 225 interposed in parallel to the charging capacitors 212, 222.

In the device of the embodiment, IGBTs are used as the switching elements 211, 221, and Free Wheeling Diodes (FWD) 211a, 221a for load current commutation are connected in parallel to the IGBTs. The switching elements 211, 221 are configured to perform an on/off operation at a predetermined timing by a control device (not illustrated). The operation of the switching elements 211, 221 will be described later in detail.

The charging capacitors 212, 222 are each implemented by connecting two capacitors in series, and this is intended to improve a withstand voltage of the capacitor. The resistors 215, 225 are intended to adjust voltages applied to the capacitors connected in series.

The resonant capacitors 213, 223 constitute parallel resonant circuits resonating at a predetermined frequency together with the magnetic stimulation coils 11, 12 connected in parallel.

The resonant circuits 21, 22 of the embodiment are configured to apply required voltages to the parallel resonant circuits (that is, resonant circuits including the magnetic stimulation coils 11, 12 and the resonant capacitors 213, 223) via the switching elements 211, 221 by electric potential differences accumulated in the charging capacitors 212, 222.

(Magnetic Stimulation Coil)

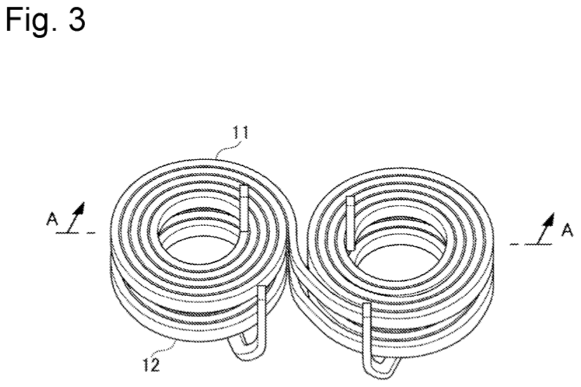

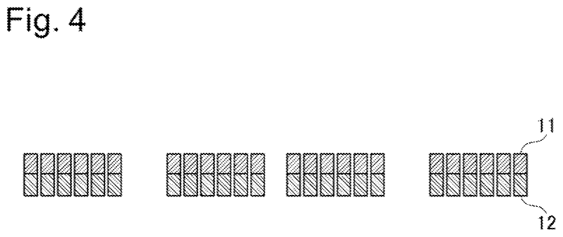

The plurality of magnetic stimulation coils 11, 12 are formed in approximately the same shape, and are adjacently disposed such that directions of magnetic fluxes generated by the applied pulse currents are matched (see FIG. 3 and FIG. 4). That is, the plurality of magnetic stimulation coils 11, 12 are disposed to be stacked such that their axial centers are approximately matched. More specifically, one of the plurality of magnetic stimulation coils 11, 12 is disposed as an upper coil 11, and the other is disposed as a lower coil 12.

The upper coil 11 and the lower coil 12 are disposed to be stacked such that a bottom surface of the upper coil 11 is overlapped with an upper surface of the lower coil 12 in a cross-sectional view of at least a part thereof (see FIG. 4). In FIG. 3, for visibility, a state where the upper coil 11 is slightly spaced from the lower coil 12 is illustrated. While both of the coils 11, 12 in the embodiment are what is called figure-eight coils, a coil having another shape may be used.

(Power Source)

As the power source 3, in this embodiment, a step-up transformer including a primary side coil 31 and a secondary side coil 32 is used. The primary side coil 31 is connected to, for example, a commercial AC power supply, and is configured to be supplied with a required electric power. The secondary side coil 32 is what is called a center-tapped coil, and is configured to supply respective required electric powers to the resonant circuits 21, 22 in one side and the other side across the center tap.

### Operation in the Embodiment

Next, an operation of the device of the embodiment having the above-described configuration will be described. Here, since both of the plurality of resonant circuits operate basically similarly, one resonant circuit 21 will be basically described below as an example.

First, assume that the switching element 211 is OFF in the initial state. In this state, when a predetermined voltage is supplied from the power source 3, the voltage rectified by the rectifier diode 214 is applied to the charging capacitors 212, and the electric charge is accumulated. Then, when the switching element 211 is turned ON by an input signal from the control device (not illustrated) at a predetermined timing, the current from the charging capacitor 212 flows in the upper coil (one magnetic stimulation coil) 11 at a resonant frequency of an LC parallel resonant circuit including the magnetic stimulation coil 11 and the resonant capacitor 213. Subsequently, when the switching element 211 is turned OFF at a predetermined timing, the status returns to the initial state. In the following, the similar operation is repeated.

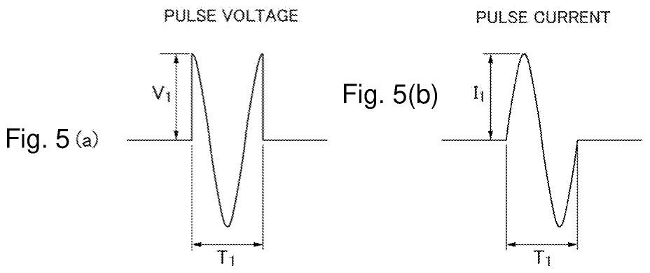

FIG. 5(a) and FIG. 5(b) illustrate an example of a voltage waveform and a current waveform applied to the coil 11. These are both sine waves, and their frequencies are determined depending on the resonant frequency of the resonant circuit. The voltage waveform and the current waveform are out of phase with each other by 90°. A time period in which the switching element 211 is ON is assumed to be T1. The time period T1 is, for example, 200 to 300 μs. However, the time period can be changed depending on the usage of the magnetic stimulation as necessary. In this example, T1 matches a cycle of the resonant frequency. While it is assumed that, in the device of this embodiment, a maximum applied voltage Vi in the positive direction to the coil 11 is 1.8 kV, and a maximum current Ii flowing in the positive direction is 7 kA, this is merely one example, and the maximum applied voltage Vi can be adjusted depending on the magnitude of the required stimulation.

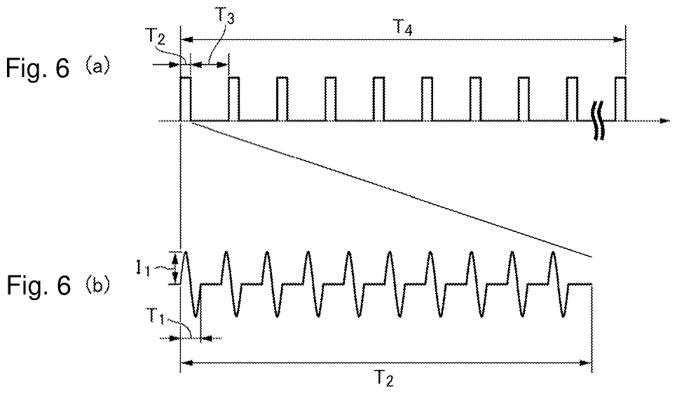

FIG. 6(a) and FIG. 6(b) illustrates an example of a treatment pattern in this embodiment. In this example, the treatment is performed during a time period (treatment time) T2 (see FIG. 6(a)), and the treatment is stopped during a next time period (downtime) T3. This operation is periodically performed. A whole treatment time T4 is, for example, from 30 minutes to 40 minutes. In one treatment time T2, by turning on/off the switching element 211, for example, a pulse current of 10 pulses per second (that is, 10 pps) is applied to the coil 11, and a variable magnetic field can be applied to the living body from the coil 11. For example, when a variable magnetic field of 3000 pulses is applied to the living body, the treatment time T4 in this example is 37.5 minutes. Obviously, this values are merely one example, and can be changed as necessary. A ratio (duty ratio) between the treatment time T2 and the downtime T3 also can be appropriately set depending on the usage. The operation of the resonant circuit 22 is similar to the above-described operation of the resonant circuit 21.

In this embodiment, since the plurality of magnetic stimulation coils 11, 12 are formed in approximately the same shape, approximately the same inductance characteristics can be obtained. Therefore, the phases of fluctuation of the magnetic fields generated from the respective coils in the resonance become approximately the same phase. Then, since the magnetic stimulation coils are adjacently disposed such that the directions of the magnetic fluxes generated by the pulse currents are matched, by mutually overlapping the generated magnetic fluxes, the magnetic flux increased to a required degree in intensity can be applied to the living body. Accordingly, there is an advantage that the current and the voltage to be applied per one of the magnetic stimulation coils 11, 12 can be reduced to be low. Then, since a low-price element, for example, an IGBT as a general-purpose product can be used as the switching elements 211, 221, an advantage of allowing the reduction of the manufacturing cost of the device to be low also can be provided.

In this embodiment, since the currents flowing in the magnetic stimulation coils 11, 12 can be reduced, the heat generation amount per one of the magnetic stimulation coils can be reduced. Accordingly, cooling measures can be facilitated, thus providing an advantage that the cooling mechanism can be simplified or eliminated.

Furthermore, since the maximum voltage and the maximum current can be reduced in each of the resonant circuits, a generated electromagnetic noise can be reduced, and as a result, measures against noise can be simplified. Insulation measures also can be simplified.

### Second Embodiment

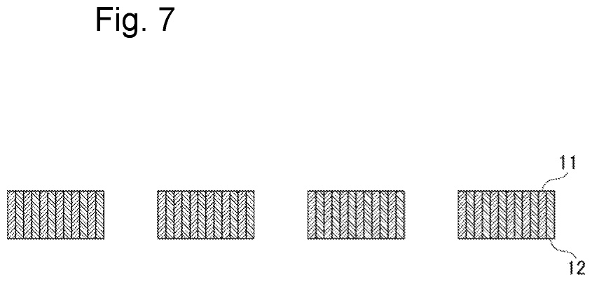

Next, a transcranial magnetic stimulator according to the second embodiment of the present invention will be described with reference to FIG. 7. In the description of the second embodiment, for the components basically in common with the device according to the first embodiment, the same reference numerals are used, thereby avoiding the overlapping description.

In the first embodiment, as the plurality of magnetic stimulation coils 11, 12, the upper coil 11 and the lower coil 12 are used. In contrast, in the second embodiment, as illustrated in FIG. 7, the plurality of magnetic stimulation coils 11, 12 are disposed such that wound wires of these coils 11, 12 are adjacent in a right-left direction (direction perpendicular to the axial center). That is, the magnetic stimulation coils 11, 12 of this embodiment are dual spiral coils concentrically stacked in a radial direction. The magnetic stimulation coils 11 and 12 are mutually insulated.

Since other configurations and the advantage of the second embodiment are similar to the first embodiment, the further detailed description will be omitted.

### Third Embodiment

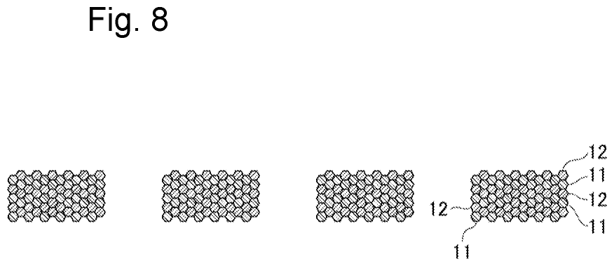

Next, a transcranial magnetic stimulator according to the third embodiment of the present invention will be described with reference to FIG. 8. In the description of the third embodiment, for the components basically in common with the device according to the first embodiment, the same reference numerals are used, thereby avoiding the overlapping description.

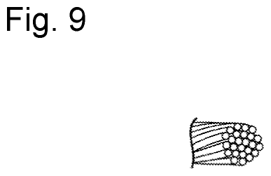

While in the first embodiment, as the plurality of magnetic stimulation coils 11, 12, the upper coil 11 and the lower coil 12 are used, in the third embodiment, as illustrated in FIG. 8, the respective wound wires of the plurality of magnetic stimulation coils 11, 12 are mutually twisted, and form a multicore wire. That is, in this embodiment, a group of core wires in the multicore wire (what is called a litz wire) constitutes one coil, and the other group of core wires constitutes the other coil. Obviously, outer peripheral surfaces of the respective core wires are insulated. In the example of FIG. 8, the core wires in the even-numbered layers from the top constitute the one magnetic stimulation coil 11, and the core wires in the odd-numbered layers constitute the other magnetic stimulation coil 12. FIG. 9 illustrates a specific example of the multicore wire. In FIG. 9, a cross-sectional shape of the whole multicore wire is a circular shape. A cross-sectional shape of the individual core wire is also a circular shape.

Since other configurations and the advantage of the third embodiment are similar to the first embodiment, the further detailed description will be omitted.

### Fourth Embodiment

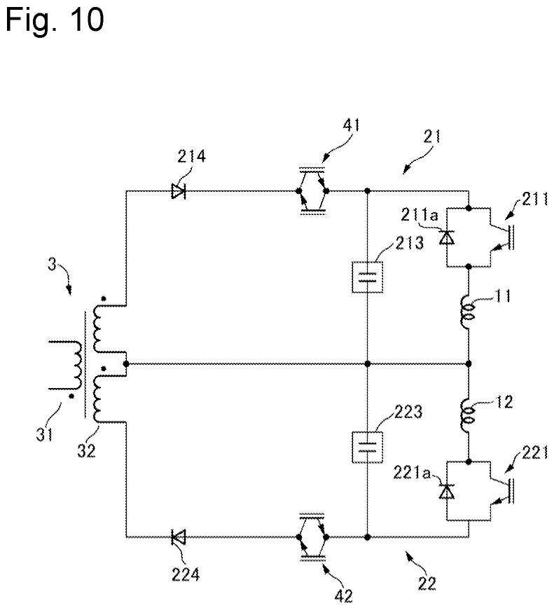

Next, a transcranial magnetic stimulator according to the fourth embodiment of the present invention will be described with reference to FIG. 10. In the description of the fourth embodiment, for the components basically in common with the device according to the first embodiment, the same reference numerals are used, thereby avoiding the overlapping description. In the fourth embodiment, the charging capacitors 212, 222 in the first embodiment are omitted, and the resonant capacitors 213, 223 double as the charging capacitors. That is, the resonant circuits 21, 22 in the fourth embodiment are LC resonant circuits including the magnetic stimulation coils 11, 12 and the resonant capacitors 213, 223.

In the fourth embodiment, between the resonant capacitors 213, 223 of the resonant circuits 21, 22 and the power source 3, second switching elements 41, 42 (see FIG. 10) that block the connection between the resonant capacitors 213, 223 and the power source 3 during the discharge of the resonant capacitors 213, 223 are disposed.

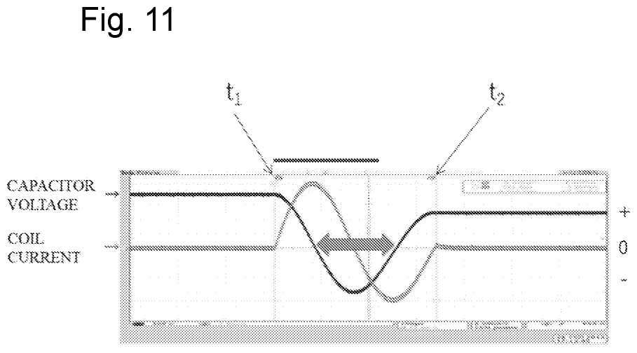

The operation of the second switching elements 41, 42 will be described further with reference to FIG. 11. This drawing illustrates an example of temporal changes of the voltages of the resonant capacitors 213, 223 and the currents flowing in the magnetic stimulation coils 11, 12 when the switching elements 211, 221 turn ON at a time t1, and turn OFF at a time t2. The voltages of the resonant capacitors 213, 223 decrease from the time t1, become negative voltages at a certain time point, and then, return to values close to the original voltage. Here, during the negative voltages (that is, during discharge of the charging voltage), parts of the currents to be flowed in the magnetic stimulation coils 11, 12 leak to the power source 3 side, and these become a loss. Then, it takes a time to recharge the resonant capacitors 213, 223, and this interferes with providing higher frequency of the magnetic stimulation pulse applied to a subject. Therefore, in this embodiment, by disposing the second switching elements 41, 42 to block the connection between the resonant capacitors 213, 223 and the power source 3 during the discharge of the resonant capacitors 213, 223, the leakage of the electric charge is reduced, thus allowing the improvement of the energy efficiency of the device. Accordingly, the leakage current in the power source 3 direction can be avoided, and consequently, the recharging time of the resonant capacitors 213, 223 is reduced, and the pulse cycle of the variable magnetic field can be reduced (that is, higher frequency can be provided). Additionally, avoiding the leakage current reduces the power consumption, thereby allowing the device to avoid the heat generation and a heat insulation structure to be simplified. In this embodiment, since a bidirectional switch is used for the second switching elements 41, 42, there is an advantage that a path for causing the charging capacitor to absorb an overvoltage generated on the wiring inductance of the device can be ensured.

Since other configurations and the advantage of the fourth embodiment are similar to the first embodiment, the further detailed description will be omitted.

### Fifth Embodiment

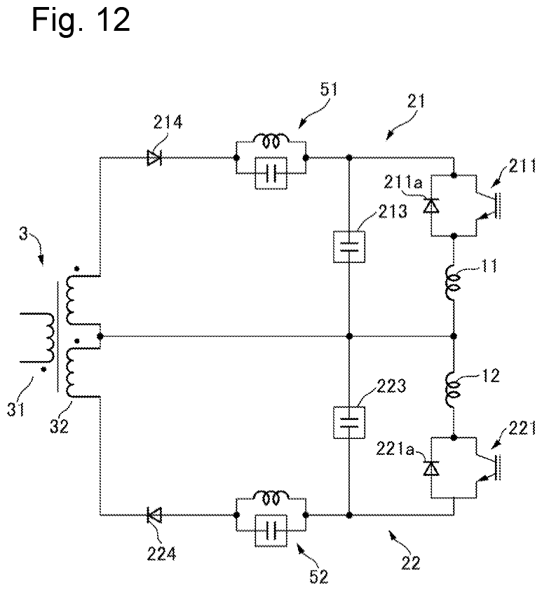

Next, a transcranial magnetic stimulator according to the fifth embodiment of the present invention will be described with reference to FIG. 12. In the description of the fifth embodiment, for the components basically in common with the device according to the fourth embodiment, the same reference numerals are used, thereby avoiding the overlapping description.

In the fifth embodiment, between the resonant capacitors 213, 223 and the power source 3, resonant impedance circuits 51, 52 including LC parallel resonant circuits are interposed. The resonant impedance circuits 51, 52 are configured to resonate at the resonant frequencies of the resonant circuits 21, 22, thereby acting as resistance components (infinite impedance in principle) higher than resistance components in non-resonance.

As described in the fourth embodiment, during the discharge of the resonant capacitors 213, 223, parts of the electric charges to be flowed in the coils 11, 12 leak to the power source 3 side. Therefore, in the fifth embodiment, the resonant impedance circuits 51, 52 suppress the current to the power source 3 side, thereby allowing the improvement of the energy efficiency of the device. Additionally, in the fifth embodiment, since the leakage current can be efficiently suppressed by only passive elements without using active elements, not only the device cost can be reduced, but also the reliability and the durability of the device can be improved. Here, while using resistive elements instead of the resonant impedance circuit can slightly reduce the leakage current, using the resonant impedance circuit provides an advantage of the high suppression effect to the leakage current.

Since other configurations and the advantage of the fifth embodiment are similar to the fourth embodiment, the further detailed description will be omitted.

### Sixth Embodiment

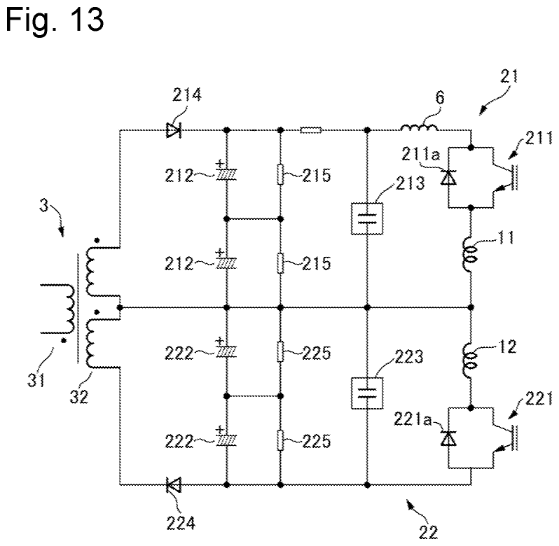

Next, a transcranial magnetic stimulator according to the sixth embodiment of the present invention will be described with reference to FIG. 13. In the description of the sixth embodiment, for the components basically in common with the device according to the first embodiment, the same reference numerals are used, thereby avoiding the overlapping description.

In the sixth embodiment, a synchronization adjustment circuit 6 for synchronizing the resonant frequencies between the respective resonant circuits is interposed in any or both of the resonant circuits. Specifically, in the example of FIG. 13, as the synchronization adjustment circuit 6, a minute inductance component interposed in the resonant circuit 21 to be in series with the magnetic stimulation coil 11 is used.

According to the sixth embodiment, by the fine adjustment of the inductance component of the resonant circuit 21, the resonant frequency of the resonant circuit 21 can be adjusted to synchronize the resonant frequencies of the respective resonant circuits. That is, with the device of this embodiment, the phases of the pulsed magnetic fluxes from the magnetic stimulation coils can be more accurately matched. Consequently, the maximum voltages and the maximum currents of the respective resonant circuits can be more suppressed.

The synchronization adjustment circuit 6 may adjust another component (for example, a capacitance component) that determines the resonant frequency. The synchronization adjustment circuit 6 may be configured to be interposed in another resonant circuit other than the resonant circuit 21, and adjust the resonant frequency of the resonant circuit.

Since other configurations and the advantage of the sixth embodiment are similar to the first embodiment, the further detailed description will be omitted.

### Seventh Embodiment

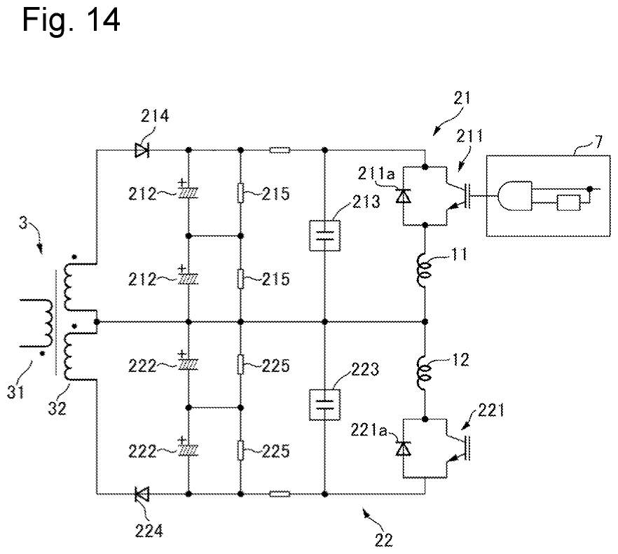

Next, a transcranial magnetic stimulator according to the seventh embodiment of the present invention will be described with reference to FIG. 14. In the description of the seventh embodiment, for the components basically in common with the device according to the first embodiment, the same reference numerals are used, thereby avoiding the overlapping description.

The device of the seventh embodiment includes a phase adjustment circuit 7 for matching phases of respective resonant currents generated in the resonant circuits 21, 22. The phase adjustment circuit 7 performs the adjustment so as to match the generation timings of the respective resonant currents generated in the resonant circuits 21, 22, thereby matching the phases of the resonant currents.

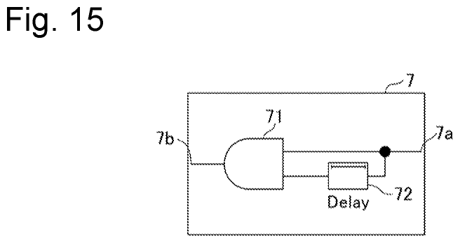

FIG. 15 illustrates an example of the phase adjustment circuit 7. The phase adjustment circuit 7 illustrated in FIG. 15 includes an AND gate 71 and a delay circuit 72. To one input terminal 7a of the AND gate 71, an input signal (ON signal) to the switching element 211 is input from the control device (not illustrated). The delay circuit 72 is configured to delay a signal to the other input terminal of the AND gate 71 corresponding to a phase shift between the resonant current of the resonant circuit 21 and the resonant current of the resonant circuit 22. An output terminal 7b of the AND gate 71 is connected to a gate of the switching element 211.

The device of this embodiment can adjust the generation timing of the resonant current by delaying the input signal to the switching element 211. That is, the adjustment can be performed in the direction decreasing the difference of the resonance start timing. Accordingly, the phases of the resonant currents generated in the resonant circuits, that is, the phases of the pulsed magnetic fluxes generated from the magnetic stimulation coils 11, 12 can be more accurately matched (that is, the difference can be decreased). Consequently, the maximum voltages and the maximum currents of the respective resonant circuits can be more suppressed.

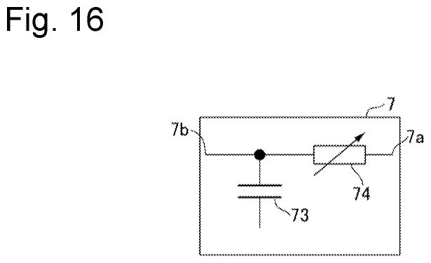

As the phase adjustment circuit 7, not limited to the example of FIG. 15, another configuration capable of adjusting the phase of the resonant current can be used. For example, as illustrated in FIG. 16, as the phase adjustment circuit 7, a capacitance element 73 and a variable resistor 74 may be used. To an input terminal 7a of this phase adjustment circuit 7, the input signal (ON signal) from the control device is input, and an output terminal 7b is connected to the gate of the switching element 211. A delay time of the circuit of FIG. 16 is determined by constants that are Cies (input capacitance) and Vth (threshold voltage) of the gate of the switching element 211, a capacitance C of the capacitance element 73 of the phase adjustment circuit 7, and a resistance value R of the variable resistor 74. Accordingly, the delay time can be controlled by adjusting the resistance value R of the variable resistor 74.

The phase adjustment circuit 7 may be connected to the resonant circuit 22 instead of the resonant circuit 21. The phase adjustment circuits 7 connected to the respective resonant circuits 21, 22 may be different each other.

Since other configurations and the advantage of the seventh embodiment are similar to the first embodiment, the further detailed description will be omitted.

### Eighth Embodiment

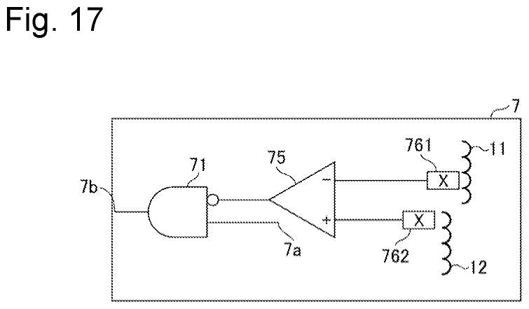

Next, a transcranial magnetic stimulator according to the eighth embodiment of the present invention will be described with reference to FIG. 17. In the description of the eighth embodiment, for the components basically in common with the device according to the seventh embodiment, the same reference numerals are used, thereby avoiding the overlapping description.

The eighth embodiment describes a further specific example of the phase adjustment circuit 7 described in the seventh embodiment. The phase adjustment circuit 7 includes a difference amplifier 75 that outputs a signal to an inverting input of an AND gate 71, and Hall elements 761, 762 that output signals to inputs of the difference amplifier 75. To another input terminal 7a of the AND gate 71, a switching signal from the control device (not illustrated) is input. The Hall element 761 is disposed in a proximity of the magnetic stimulation coil 11, and configured to detect an intensity of a magnetic field generated from the magnetic stimulation coil 11 as a voltage value. Similarly, the Hall element 762 is disposed in a proximity of the magnetic stimulation coil 12, and configured to detect an intensity of a magnetic field generated from the magnetic stimulation coil 12 as a voltage value.

In the device of the eighth embodiment, when there is no difference between the signals from the Hall elements 761, 762 (that is, when the phases of the magnetic field intensities are matched), an IGBT signal (that is, a switching signal) from the control device is directly input to the switching element 211. When there is a difference between the signals from the Hall elements 761, 762 (that is, when the phases of the magnetic field intensities are shifted), the IGBT signal (that is, a switching signal) from the control device is not input to the switching element 211, and becomes a state of hold. This allows automatically adjusting the resonance start timing in the resonant circuit 21.

Since other configurations and the advantage of the eighth embodiment are similar to the seventh embodiment, the further detailed description will be omitted.

### Ninth Embodiment

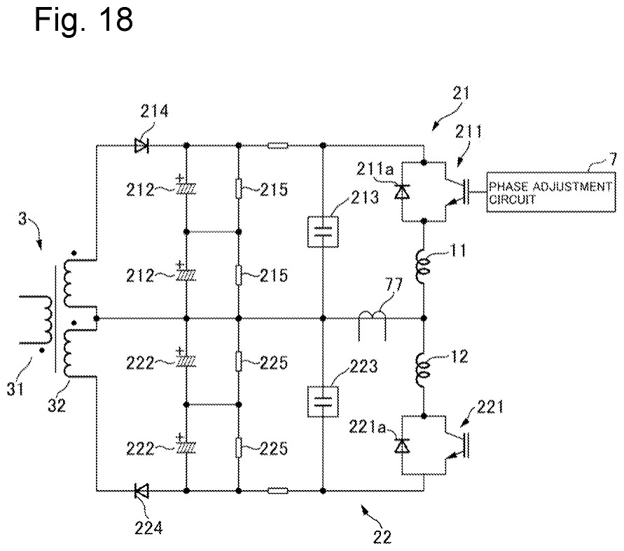

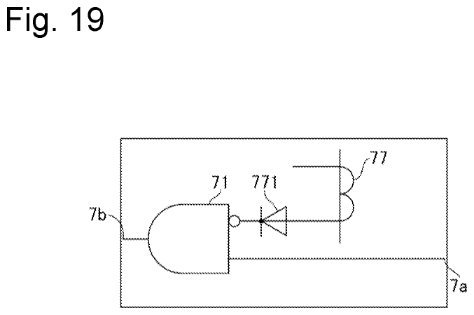

Next, a transcranial magnetic stimulator according to the ninth embodiment of the present invention will be described with reference to FIG. 18 and FIG. 19. In the description of the ninth embodiment, for the components basically in common with the device according to the seventh embodiment, the same reference numerals are used, thereby avoiding the overlapping description.

The ninth embodiment describes a further specific example of the phase adjustment circuit 7 described in the seventh embodiment. A current sensing element 77 is interposed in a common wiring part of the resonant circuits 21, 22 of the ninth embodiment (see FIG. 18). The current sensing element 77 detects a current value of the common wiring. In the phase adjustment circuit 7 of the ninth embodiment, a signal from the current sensing element 77 is input to an inverting input of an AND gate 71 via a rectifier diode 771 (see FIG. 19).

In the device of the ninth embodiment, when there is no phase difference between the respective resonant currents flowing in the resonant circuits 21, 22, the current does not flow in the common wiring part of these circuits. Accordingly, an IGBT signal (that is, a switching signal) from the control device is directly input to the switching element 211. When there is a phase difference between the respective resonant currents flowing in the resonant circuits 21, 22, the current corresponding to the phase difference flows in the common wiring part of these circuits. Then, the IGBT signal (that is, a switching signal) from the control device is not input to the switching element 211, and becomes a state of hold. This allows automatically adjusting the resonance start timing in the resonant circuit 21.

Since other configurations and the advantage of the ninth embodiment are similar to the seventh embodiment, the further detailed description will be omitted.

### Tenth Embodiment

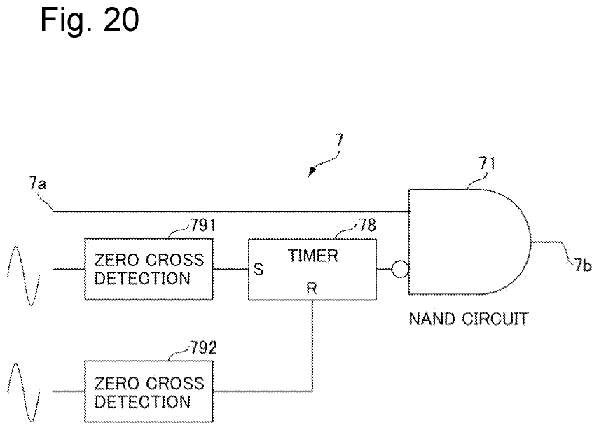

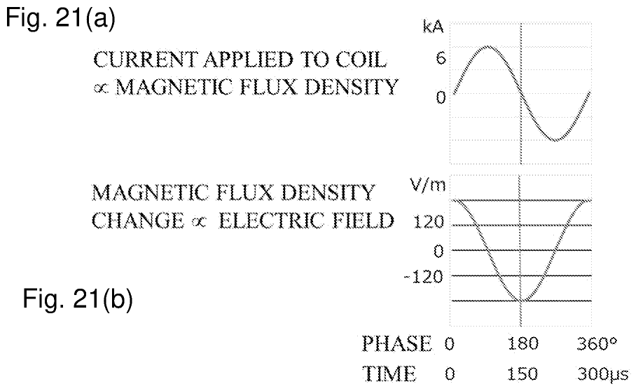

Next, a transcranial magnetic stimulator according to the tenth embodiment of the present invention will be described with reference to FIG. 20 and FIG. 21(a) and FIG. 21(b). In the description of the tenth embodiment, for the components basically in common with the device according to the seventh embodiment, the same reference numerals are used, thereby avoiding the overlapping description.

The tenth embodiment describes another example of the phase adjustment circuit 7 described in the seventh embodiment. The phase adjustment circuit 7 is configured to match the phases of the resonant currents by performing the adjustment such that maximum points of change rates (that is, dI/dt) of respective resonant currents generated in the resonant circuits 21, 22 are matched (that is, such that the difference is decreased).

The phase adjustment circuit 7 of the tenth embodiment includes a timer 78 connected to an inverting input of an AND gate 71, and zero cross detectors 791, 792 connected to the timer 78. The zero cross detector 791 is configured to detect a zero cross point in a sine waveform of the resonant current flowing in the resonant circuit 21. Similarly, the zero cross detector 792 is configured to detect a zero cross point in a sine waveform of the resonant current flowing in the resonant circuit 22. When the zero cross points are matched, an IGBT signal (that is, a switching signal) from the control device is directly input to the switching element 211. When the zero cross points are shifted, the shift is measured by the timer 78, and by the measured period, a time point of the next resonance start in one resonant circuit can be shifted (that is, delayed) by the time period. This allows matching the phases of the respective resonant currents generated in the resonant circuits 21, 22.

FIG. 21(a) and FIG. 21(b) illustrates a relation between the current applied to the coil and an electric field generated by the coil. The magnetic flux density of the coil is proportionate to the current value, and the electric field is proportionate to the change of the magnetic flux density. When the change rate (dI/dt) of the current applied to the coil is maximum, the electric field generated by the coil becomes maximum (see FIG. 21(b)). Accordingly, by matching the maximum points of the change rates of the currents, it can be attempted to make the electric field by a plurality of coils maximum. Therefore, a high treatment effect can be expected.

Since other configurations and the advantage of the tenth embodiment are similar to the seventh embodiment, the further detailed description will be omitted.

The contents of the present invention are not limited to the above-described embodiments. In the present invention, various kinds of changes can be made on the specific configurations within the scope of the claims.

For example, while the example of using the two resonant circuits is described in each of the above-described embodiments, it is possible to use three or more resonant circuits, thereby driving corresponding magnetic stimulation coils by the respective resonant circuits. In this case, the resonant circuits are each in parallel to the power source 3.

While the configuration in which the currents in the same phase are applied to the plurality of magnetic stimulation coils 11, 12 is described in each of the above-described embodiments, it is possible to apply a current in the opposite phase to invert the direction of the magnetic field, thereby canceling the magnetic field (ideally, making the magnetic field intensity zero). This allows the use as a sham stimulation coil for a clinical study.

### DESCRIPTION OF REFERENCE SIGNS

3 . . . Power source
6 . . . Synchronization adjustment circuit
7 . . . Phase adjustment circuit
11 . . . One magnetic stimulation coil (upper coil)
12 . . . Other magnetic stimulation coil (lower coil)
21, 22 . . . Resonant circuit
211, 221 . . . Switching element
212, 222 . . . Charging capacitor
213, 223 . . . Resonant capacitor
214, 224 . . . Diode
215, 225 . . . Resistor
41, 42 . . . Second switching element
51, 52 . . . Resonant impedance circuit
100 . . . Device main body
200 . . . Adjustment mechanism
210 . . . Coil holder

1. A transcranial magnetic stimulator comprising:
a plurality of resonant circuits including a plurality of magnetic stimulation coils for stimulating a living body by applying variable magnetic fields to an inside of the living body, the plurality of resonant circuits applying respective pulse currents to the plurality of magnetic stimulation coils to generate the variable magnetic fields; and
a power source that supplies an electric power to the plurality of resonant circuits, wherein
the plurality of resonant circuits are connected in parallel to the power source, and therefore, the plurality of magnetic stimulation coils are also connected in parallel to the power source, and
the plurality of magnetic stimulation coils are formed in approximately a same shape, and adjacently disposed such that directions of magnetic fluxes generated by the pulse currents are matched.

2. The transcranial magnetic stimulator according to claim 1, wherein
each of the plurality of resonant circuits includes a switching element that controls an application timing of the pulse current to the magnetic stimulation coil.

3. The transcranial magnetic stimulator according to claim 1, wherein
the plurality of magnetic stimulation coils are disposed to be stacked such that axial centers of the plurality of magnetic stimulation coils are approximately matched.

4. The transcranial magnetic stimulator according to claim 3, wherein
one of the magnetic stimulation coils is an upper coil, and another is a lower coil, and
the upper coil and the lower coil are disposed to be stacked such that a bottom surface of the upper coil is overlapped with an upper surface of the lower coil in a cross-sectional view of at least a part of the upper coil and the lower coil.

5. The transcranial magnetic stimulator according to claim 1, wherein
respective wound wires of the plurality of magnetic stimulation coils are mutually twisted, and form a multicore wire.

6. The transcranial magnetic stimulator according to claim 1, wherein
the resonant circuit includes a resonant capacitor that accumulates an electric charge supplied from the power source,
a second switching element is disposed between the resonant capacitor and the power source, and the second switching element blocks a connection between the resonant capacitor and the power source during discharge of the resonant capacitor to suppress a leakage current to the power source.

7. The transcranial magnetic stimulator according to claim 1, wherein
the resonant circuit includes a resonant capacitor that accumulates an electric charge supplied from the power source, and
a resonant impedance circuit is interposed between the resonant capacitor and the power source, and the resonant impedance circuit acts as a resistance component higher than a resistance component in non-resonance by resonating at a resonant frequency of the resonant circuit to suppress a leakage current to the power source.

8. The transcranial magnetic stimulator according to claim 1, wherein
any or all of the plurality of resonant circuits include a synchronization adjustment circuit for synchronizing the resonant frequency of each of the resonant circuits.

9. The transcranial magnetic stimulator according to claim 1, comprising
a phase adjustment circuit for matching phases of respective resonant currents generated in the plurality of resonant circuits.

10. The transcranial magnetic stimulator according to claim 9, wherein
the phase adjustment circuit is configured to match the phases of the resonant currents by performing an adjustment so as to match generation timings of the respective resonant currents generated in the plurality of resonant circuits.

11. The transcranial magnetic stimulator according to claim 9, wherein
the phase adjustment circuit is configured to match the phases of the resonant currents by performing an adjustment so as to match maximum points of change rates of the respective resonant currents generated in the plurality of resonant circuits.

12. A transcranial magnetic stimulator comprising:
a plurality of resonant circuits including a plurality of magnetic stimulation coils for stimulating a living body by applying variable magnetic fields to an inside of the living body, the plurality of resonant circuits applying respective pulse currents to the plurality of magnetic stimulation coils to generate the variable magnetic fields; and
a power source that supplies an electric power to the plurality of resonant circuits, wherein
the plurality of resonant circuits are connected in parallel to the power source, and therefore, the plurality of magnetic stimulation coils are also connected in parallel to the power source, and
the transcranial magnetic stimulator further comprises a phase adjustment circuit for matching phases of respective resonant currents generated in the plurality of resonant circuits.

13. The transcranial magnetic stimulator according to claim 12, wherein
the phase adjustment circuit is configured to match the phases of the resonant currents by performing an adjustment so as to match maximum points of change rates of the respective resonant currents generated in the plurality of resonant circuits.
